(19) United States
(12) Patent Application Publication
(10) Pub. No.: US 2017/0056365 A1
(43) Pub. Date: Mar. 2, 2017

(54) DAPAGLIFLOZIN COMPOSITIONS

(71) Applicant: SUN PHARMACEUTICAL INDUSTRIES LIMITED, Mumbai, Maharashtra (IN)

(72) Inventors: Suhani SINHA, Gurgaon (IN); Roshan Lal SANDAL, Firozpur (IN); Ravi KOCHHAR, Gurgaon (IN)

(73) Assignee: SUN PHARMACEUTICAL INDUSTRIES LIMITED, Mumbai, Maharashitra (IN)

(21) Appl. No.: 15/121,888

(22) PCT Filed: Feb. 27, 2015
(86) PCT No.: PCT/IB2015/051479

§ 371 Date: Aug. 26, 2016

(30) Foreign Application Priority Data
Feb. 28, 2014 (IN) 583/DEL/2014

(51) Int. Cl. A61K 31/351; A61K 9/20

(52) U.S. Cl. CPC A61K 31/351; A61K 9/2095; A61K 9/2081; A61K 9/2027; A61K 9/2054; A61K 9/2013; A61K 9/2009

(57) ABSTRACT

The present invention relates to pharmaceutical compositions comprising a solid dispersion of dapagliflozin and one or more pharmaceutically acceptable excipients, and processes for their preparation. It further relates to a method of treating diabetes using said pharmaceutical compositions.

### FIELD OF THE INVENTION

The present invention relates to pharmaceutical compositions comprising a solid dispersion of dapagliflozin and one or more pharmaceutically acceptable excipients, and processes for their preparation. It further relates to a method of treating diabetes using said pharmaceutical compositions.

### BACKGROUND OF THE INVENTION

Dapagliflozin is a SGLT2 inhibitor, chemically known as (2S,3R,4R,5S,6R)-2-[4-chloro-3-(4-ethoxybenzyl)phenyl]-6-(hydroxymethyl)tetrahydro-2H-pyran-3,4,5-triol. The commercially available formulations of dapagliflozin contain the propanediol (propylene glycol) monohydrate solvate of dapagliflozin as the active ingredient. The drug substance is crystalline in nature. In contrast, dapagliflozin base is present in amorphous form.

U.S. Pat. No. 6,515,117 discloses dapagliflozin as a compound. U.S. Pat. No. 7,919,598 discloses the (S)-propylene glycol solvate of dapagliflozin and processes of preparation thereof.

U.S. Pat. Nos. 7,851,502, 8,221,786, and 8,361,972 disclose pharmaceutical compositions comprising dapagliflozin or dapagliflozin propylene glycol hydrate and specific excipients in given amounts. U.S. Pat. No. 8,221,786 further discloses pharmaceutical compositions in the form of a stock granulation comprising dapagliflozin propylene glycol hydrate and excipients in given amounts.

PCT Publication No. WO 2012/163546 discloses pharmaceutical compositions comprising cyclodextrin and dapagliflozin, preferably as an inclusion complex.

The dapagliflozin base is hygroscopic in nature. It absorbs moisture and forms sticky lumps which are difficult to process and handle, and which may ultimately lead to content uniformity issues in the dosage form. The low solubility and stability of dapagliflozin base as compared to its solvates may lead to poor bioavailability of the drug.

Therefore, there exists a need to provide a stable form of dapagliflozin base that is readily bioavailable and can be further processed to form stable compositions.

The inventors of the present invention have found that a solid dispersion of dapagliflozin is more stable, has superior solubility, and has better processing abilities as compared to dapagliflozin base. Further, the solid dispersion can be conveniently formulated into various dosage forms.

### SUMMARY OF THE INVENTION

The present invention relates to pharmaceutical compositions comprising a solid dispersion of dapagliflozin and one or more pharmaceutically acceptable excipients. The solid dispersion comprises dapagliflozin and a carrier, wherein the dapagliflozin is dispersed or dissolved in the carrier.

The present invention also includes different processes for the preparation of said pharmaceutical composition and a method of treating diabetes by administering said pharmaceutical composition.

### DETAILED DESCRIPTION OF THE INVENTION

A first aspect of the present invention provides a pharmaceutical composition comprising a solid dispersion of dapagliflozin and one or more pharmaceutically acceptable excipients.

According to one embodiment of this aspect, the solid dispersion comprises dapagliflozin and a carrier, wherein the dapagliflozin is dispersed or dissolved in the carrier.

According to another embodiment of this aspect, the carrier comprises a hydrophilic polymer and optionally one or more pharmaceutically acceptable excipients.

According to another embodiment of this aspect, the weight ratio of dapagliflozin to the carrier is about 1:0.1 to about 1:100.

According to another embodiment of this aspect, the pharmaceutically acceptable excipients are selected from the group comprising diluents, binders, disintegrants, lubricants, glidants, stabilizers, surfactants, solubility enhancers, coloring agents, flavoring agents, and mixtures thereof.

According to another embodiment of this aspect, the solid dispersion is prepared by a solvent method.

According to another embodiment of this aspect, the solid dispersion is prepared by a hot-melt extrusion method.

According to another embodiment of this aspect, the pharmaceutical composition may be in the form of caplets, pills, mini-tablets, granules, pellets, tablets, or capsules.

According to another embodiment of this aspect, the pharmaceutical composition is film-coated.

A second aspect of the present invention provides a process for the preparation of a pharmaceutical composition comprising a solid dispersion of dapagliflozin and one or more pharmaceutically acceptable excipients, wherein the process comprises:
(a) dissolving a carrier in a solvent to obtain a solution;
(b) dispersing dapagliflozin in the solution of step (a) to obtain a dispersion;
(c) blending a diluent and optionally, a binder;
(d) granulating the blend of step (c) with the dispersion of step (b) to obtain drug coated granules, followed by drying of the granules;
(e) blending the dried granules of step (d) with a disintegrant, a lubricant, and/or a glidant; and
(f) compressing the lubricated granules of step (e) into tablets or filling into capsules.

The process of the second aspect of the invention may consist essentially of forming granules by dissolving a carrier in a solvent to obtain a solution, dispersing the dapagliflozin in the solution to obtain a dispersion, and granulating the dispersion with a blend of a diluent and an optional binder to form granules. The process may then further include the steps of drying the granules and then blending the dried granules with one or more of a disintegrant, lubricant and glidant to form lubricated granules. The lubricated granules may then be compressed into a tablet or filled into a capsule.

The process of the second aspect of the invention may consist of forming granules by dissolving a carrier in a solvent to obtain a solution, dispersing the dapagliflozin in the solution to obtain a dispersion, and granulating the dispersion with a blend of a diluent and an optional binder to form granules. The granules thus prepared may be further processed by drying the granules and blending the dried granules with one or more of a disintegrant, lubricant and glidant to lubricate the granules. The lubricated granules may then be compressed into a tablet or filled into a capsule.

According to one embodiment of this aspect, the carrier is a hydrophilic polymer.

A third aspect of the present invention provides a process for the preparation of a pharmaceutical composition comprising a solid dispersion of dapagliflozin and one or more pharmaceutically acceptable excipients, wherein the process comprises:
(a) dissolving dapagliflozin in a carrier;
(b) blending a diluent and optionally, a binder;
(c) granulating the blend of step (b) with the solution of step (a) to obtain drug coated granules, followed by drying of the granules;
(d) blending the dried granules of step (c) with a disintegrant, a lubricant, and/or a glidant; and
(e) compressing the lubricated granules of step (d) into tablets or filling into capsules.

The process of the third aspect of the invention may consist essentially of forming granules by dissolving dapagliflozin in a carrier to obtain a solution and granulating the solution with a blend of a diluent and an optional binder to form granules. The process may then further include the steps of drying the granules, and then blending the dried granules with one or more of a disintegrant, lubricant and glidant to form lubricated granules. The lubricated granules may then be compressed into a tablet or filled into a capsule.

The process of the third aspect of the invention may consist of forming granules by dissolving dapagliflozin in a carrier to obtain a solution and granulating the solution with a blend of a diluent and an optional binder to form granules. The granules thus prepared may be further processed by drying the granules and blending the dried granules with one or more of a disintegrant, lubricant and glidant to lubricate the granules. The lubricated granules may then be compressed into a tablet or filled into a capsule.

According to one embodiment of this aspect, the carrier is a hydrophilic polymer.

A fourth aspect of the present invention provides a process for the preparation of a pharmaceutical composition comprising a solid dispersion of dapagliflozin and one or more pharmaceutically acceptable excipients, wherein the process comprises:
(a) blending dapagliflozin, a carrier, and a glidant in a rapid mixer granulator;
(b) loading the blend of step (a) into a hot-melt extruder to obtain extrudates;
(c) milling the extrudates of step (b) and blending the milled extrudates with a diluent, a binder, a disintegrant, a glidant, and/or a lubricant; and
(d) compressing the blend of step (c) into tablets or filling into capsules.

The process of the fourth aspect of the invention may consist essentially of forming extrudates by blending dapagliflozin, a carrier, and a glidant in a rapid mixer granulator to form a blend and then loading the blend of into a hot-melt extruder to obtain extrudates. The process may then further include the steps of milling the extrudates, and then blending the milled extrudates with one or more of a diluent, binder, disintegrant, lubricant and glidant to form a lubricated blend. The lubricated blend may then be compressed into a tablet or filled into a capsule.

The process of the fourth aspect of the invention may consist of forming extrudates by blending dapagliflozin, a carrier, and a glidant in a rapid mixer granulator to form a blend and then loading the blend of into a hot-melt extruder to obtain extrudates. The process may then further include the steps of milling the extrudates, and then blending the milled extrudates with one or more of a diluent, binder, disintegrant, lubricant and glidant to form a lubricated blend. The lubricated blend may then be compressed into a tablet or filled into a capsule.

According to one embodiment of this aspect, the carrier is a hydrophilic polymer.

A fifth aspect of the present invention provides a method of treating diabetes by administering a pharmaceutical composition comprising a solid dispersion of dapagliflozin and one or more pharmaceutically acceptable excipients.

According to one embodiment of this aspect, the method of treatment further comprises administration of an additional anti-diabetic agent.

The term “dapagliflozin,” as used herein, refers to dapagliflozin base. The dapagliflozin base is hygroscopic in nature, which is less stable and has low solubility as compared to its solvates. It quickly absorbs moisture, degrades, discolors, and causes sticking problems. Further, absorption of moisture results in lump formation which may ultimately lead to content uniformity issues in the dosage form. Hence, the pharmaceutical composition of the present invention is prepared in the absence of water.

The term “pharmaceutically acceptable excipients,” as used herein, includes diluents, binders, disintegrants, lubricants, glidants, stabilizers, surfactants, solubility enhancers, coloring agents, flavoring agents, and mixtures thereof.

The term “about,” as used herein, refers to any value which lies within the range defined by a variation of up to ±10% of the value.

The term “solid dispersion,” as used herein, refers to a group of solid products consisting of at least two different components, generally a carrier and a drug. The carrier can be crystalline or amorphous. The drug can be dispersed or dissolved in the carrier. The solid dispersion of dapagliflozin is more stable, has superior solubility, and better processing abilities as compared to dapagliflozin base.

The solid dispersion of dapagliflozin may be prepared by dissolving or dispersing the dapagliflozin base in the carrier. The carrier used for forming the solid dispersion comprises a hydrophilic polymer and, optionally, one or more pharmaceutically acceptable excipients. The weight ratio of the dapagliflozin to the carrier is in the range of about 1:0.1 to about 1:100; particularly in the range of about 1:0.25 to about 1:20; more particularly in the range of about 1:0.5 to about 1:10. The solid dispersion of the present invention may be prepared by a hot-melt extrusion method, by a solvent method, or by a combination of both.

In the solvent method, the solid dispersion of the dapagliflozin is loaded on to the excipients; in the hot-melt extrusion method, the solid dispersion of the dapagliflozin is converted to a particulate form. In the solvent method, the drug is dissolved/dispersed in the carrier or the drug along with the carrier is dissolved/dispersed in a solvent. The solution or dispersion is then used for granulating the blend of one or more pharmaceutically acceptable excipients. The granules are then dried to obtain the solid dispersion of the drug loaded on to the excipients. In an alternate method, the drug is dissolved/dispersed in the carrier or the drug along with the carrier is dissolved/dispersed in a solvent. The solvent is then evaporated by freeze drying or spray drying. As the solvent evaporates, supersaturation occurs, followed by simultaneous precipitation of both the carrier and the drug in solid form. The resulting precipitate, which has the drug dissolved or suspended in a carrier, is then dried to produce a solid dispersion of the invention. In the hot-melt extrusion method, the drug and the carrier are loaded in a hot-melt extruder and the resulting extrudates are milled to obtain a solid dispersion in a particulate form.

Suitable hydrophilic polymers are selected from the group comprising cellulose or derivatives thereof, e.g., methyl cellulose, hydroxypropylmethyl cellulose, hydroxypropyl cellulose, hydroxypropylmethyl cellulose phthalate, hydroxypropylmethyl cellulose acetate succinate, carboxymethylethyl cellulose, carboxylmethyl cellulose sodium, and hydroxyethyl cellulose; synthetic polymers, e.g., polyethylene glycol (e.g., Macrogol 400, Macrogol 1500, Macrogol 4000, and Macrogol 6000), polyvinyl alcohol, polyvinylpyrrolidone, polyvinylacetal diethylaminoacetate, acrylic polymers, and carboxylvinyl polymer; natural polymers or sugars, e.g., gum arabic, sodium alginate, propylene glycol alginate, agar, gelatin, tragacanth, and xanthan gum; and mixtures thereof.

Suitable solvents used for the preparation of the solid dispersion may be a single solvent or mixture of solvents, and the order of dissolution or dispersion of dapagliflozin with the carrier in the solvent may be varied. Particularly, the solvent is an organic solvent. Suitable solvents are selected from the group comprising alcohols, e.g., methanol, ethanol, and isopropyl alcohol; carboxylic acids, e.g., formic acid, acetic acid, and propionic acid; halogenated hydrocarbons, e.g., dichloromethane, dichloroethane, and methylene chloride; ketones, e.g., acetone, dimethyl ketone, ethyl methyl ketone, and methyl iso-butyl ketone; amides, e.g., N,N-dimethylformamide and N,N-dimethylacetamide; sulphoxides, e.g., dimethyl sulfoxide and diethyl sulphoxide; ethers, e.g., diethyl ether, ethyl methyl ether, di-isopropyl ether, tetrahydrofuran, and 1,4-dioxane; cyclic ethers, e.g., tetrahydrofuran; and mixtures thereof.

Suitable diluents are selected from the group comprising lactose, e.g., lactose anhydrous and lactose monohydrate; cellulose, e.g., microcrystalline cellulose, co-processed microcrystalline cellulose, and powdered cellulose; starch, e.g., pregelatinized starch, maize starch, rice starch, potato starch, and wheat starch; sugar alcohols, e.g., mannitol, sorbitol, xylitol, and erythritol; inorganic salts, e.g., calcium carbonate, calcium phosphate, calcium sulfate, dibasic calcium phosphate, dibasic calcium phosphate anhydrate, dibasic calcium phosphate dihydrate, and tribasic calcium phosphate; and mixtures thereof. The diluents may act as a binder.

Suitable binders are selected from the group comprising povidone, copovidone, hydroxypropylmethyl cellulose, hydroxypropyl cellulose, hydroxyethyl cellulose, methyl cellulose, ethyl cellulose, carboxymethyl cellulose sodium, xanthan gum, gum acacia, gum arabic, tragacanth, sorbitol, dextrose, sucrose, lactose, mannitol, gelatin, pullulan, sodium alginate, propylene glycol, polyvinyl alcohol, corn starch, modified corn starch, maize starch, pregelatinized starch, methacrylates, carboxyvinyl polymers, waxes, and mixtures thereof.

Suitable disintegrants are selected from the group comprising croscarmellose sodium, hydroxypropyl cellulose, crospovidone, low substituted hydroxypropyl cellulose, microcrystalline cellulose, carboxymethyl cellulose sodium, carboxymethyl cellulose calcium, gums, alginic acid or alginates, starch, corn starch, pregelatinized starch, modified starch, sodium starch glycolate, carboxymethyl starch, polyacrylates, and mixtures thereof.

Suitable lubricants are selected from the group comprising stearic acid, magnesium stearate, calcium stearate, zinc stearate, sodium stearyl fumarate, polyethylene glycol, talc, hydrogenated vegetable oils, fatty acids, waxes, and mixtures thereof.

Suitable glidants or anti-sticking agents are selected from the group comprising talc, silicon dioxide, colloidal silicon dioxide (colloidal anhydrous silica), calcium silicate, magnesium silicate, hydrated silica, and mixtures thereof.

Suitable stabilizers are selected from the group comprising cellulose derivatives, e.g., hydroxypropylmethyl cellulose, ethyl cellulose, hydroxyethyl cellulose, hydroxypropylmethyl cellulose phthalate, carboxymethyl cellulose sodium, and carboxymethyl cellulose calcium; vinyl polymers, e.g., polyvinylpyrrolidone, copovidone, and polyvinyl alcohol; polyethylene glycol; block copolymers of ethylene oxide and/or propylene oxide; gums, e.g., xanthan gum, gum arabic and/or derivatives thereof; pectins; alginates; acacia; tragacanth and/or derivatives thereof; carrageenans; agar and/or derivatives thereof; polysaccharides from microbiological sources; starches; arabinogalactans; galactomannans; dextrans; and mixtures thereof. The stabilizers may also act as crystallization inhibitors.

Suitable surfactants are selected from the group comprising sodium lauryl sulfate, sodium dodecyl sulfate, ammonium lauryl sulfate, benzalkonium chloride, alkyl poly(ethylene oxide), copolymers of poly(ethylene oxide) and poly(propylene oxide) commercially called as poloxamers or poloxamines, polyvinyl alcohol (PVA), fatty alcohols, polyoxyethylene alkyl ether, polyoxyethylene alkylaryl ether, polyethylene glycol fatty acid ester, alkylene glycol fatty acid mono ester, sucrose fatty acid ester, sorbitan fatty acid mono ester, sorbitol mono laurate (Span® 20 or Span® 80), polyoxyethylene sorbitan fatty acid ester (polysorbates), and mixtures thereof.

Suitable solubility enhancers are selected from the group comprising sodium lauryl sulfate, polyethylene glycol, propylene glycol, glycerol, glycerol monostearate, glycerol behenate, triglycerides, mono-alcohols, higher alcohols, dimethylsulfoxide, dimethylformamide, N,N-dimethylacetamide, N-methyl-2-pyrrolidone, N-(2-hydroxyethyl) pyrrolidone, 2-pyrrolidone, and mixtures thereof.

Suitable coloring agents and flavoring agents are selected from any FDA approved colors and flavors for oral use.

The composition of the present invention may be in the form of caplets, pills, mini-tablets, granules, pellets, tablets, or capsules. The pharmaceutical composition of the present invention may further be film-coated using techniques well known in the art such as spray coating in a conventional coating pan or a fluidized bed processor or dip coating. The film coat comprises film-forming polymers and one or more coating additives.

Suitable film-forming polymers are selected from the group comprising cellulose derivatives, e.g., methyl cellulose, hydroxymethyl cellulose, hydroxyethyl cellulose, hydroxypropyl cellulose, hydroxymethylethyl cellulose, hydroxypropylmethyl cellulose, sodium carboxymethyl cellulose, and ethyl cellulose; vinyl polymers, e.g., polyvinylpyrrolidones; acrylic polymers; and mixtures thereof. Alternatively, commercially available coating compositions comprising film-forming polymers marketed under various trade names, e.g., Opadry®, may be used for coating.

The coating additives comprise one or more of plasticizers, glidants or flow regulators, lubricants, coloring agents, and opacifiers.

Suitable plasticizers are selected from the group comprising castor oil, diacetylated monoglycerides, dibutyl sebacate, diethyl phthalate, glycerin, polyethylene glycols, propylene glycols, triacetin, triethyl citrate, and mixtures thereof. An opacifier such as titanium dioxide may also be present in the coating.

Examples of solvents used for preparing the coating solution are selected from methyl alcohol, ethyl alcohol, isopropyl alcohol, n-butyl alcohol, acetone, acetonitrile, chloroform, methylene chloride, and mixtures thereof.

The composition of the present invention is used for treating diabetes. The pharmaceutical composition of the present invention may be administered in combination with other anti-diabetic agents.

The present invention is illustrated below by reference to the following examples. However, one skilled in the art will appreciate that the specific methods and results discussed are merely illustrative of the invention, and not to be construed as limiting the invention, as many variations thereof are possible without departing from the spirit and scope of the invention.

### EXAMPLES

### Example 1

| | |
|---|---|
| Ingredients | Percent (%) w/w |

Part A (Drug Coated Granules)

| | |
|---|---|
| Dapagliflozin base | 4.05 |
| Polyethylene glycol | 8.09 |
| Isopropyl alcohol | q.s. |
| Methylene chloride | q.s. |
| Lactose anhydrous | 20.23 |
| Microcrystalline cellulose | 40.45 |

Part B

| | |
|---|---|
| Drug Coated Granules of Part A | 72.82 |
| Microcrystalline cellulose | 18.20 |
| Crospovidone | 4.05 |
| Colloidal silicon dioxide | 1.21 |
| Magnesium stearate | 0.81 |

Film coating

| | |
|---|---|
| Opadry ® coating | 2.91 |

### Procedure:

1. Lactose anhydrous and intragranular microcrystalline cellulose were blended.
2. Polyethylene glycol was dissolved in a solvent mixture (isopropyl alcohol:methylene chloride mixture (1:1.5 ratio)), followed by the dispersion of dapagliflozin base while stirring.
3. The material of step 1 was granulated with the dispersion of step 2, followed by drying of the granules.
4. The dried granules of step 3 were blended with extragranular microcrystalline cellulose and crospovidone, followed by the addition of colloidal silicon dioxide and magnesium stearate.
5. The blend of step 4 was compressed into tablets.
6. The tablets of step 5 were coated with Opadry®.

### Example 2

| | |
|---|---|
| Ingredients | Percent (%) w/w |

Part A (Drug Coated Granules)

| | |
|---|---|
| Dapagliflozin base | 4.05 |
| Polyethylene glycol | 4.05 |
| Hydroxypropylmethyl cellulose | 4.05 |
| Isopropyl alcohol | q.s. |
| Methylene chloride | q.s. |
| Lactose anhydrous | 20.23 |
| Microcrystalline cellulose | 40.44 |

Part B

| | |
|---|---|
| Drug Coated Granules of Part A | 72.82 |
| Microcrystalline cellulose | 18.20 |
| Crospovidone | 4.05 |
| Colloidal silicon dioxide | 1.21 |
| Magnesium stearate | 0.81 |

Film coating

| | |
|---|---|
| Opadry ® coating | 2.91 |

### Procedure:

1. Lactose anhydrous and intragranular microcrystalline cellulose were blended.
2. Polyethylene glycol and hydroxypropylmethyl cellulose were dissolved in a solvent mixture (isopropyl alcohol:methylene chloride mixture (1:1.5 ratio)), followed by the dispersion of dapagliflozin base while stirring.
3. The material of step 1 was granulated with the dispersion of step 2, followed by drying of the granules.
4. The dried granules of step 3 were blended with extragranular microcrystalline cellulose and crospovidone, followed by the addition of colloidal silicon dioxide and magnesium stearate.
5. The blend of step 4 was compressed into tablets.
6. The tablets of step 5 were coated with Opadry®.

### Example 3

| | |
|---|---|
| Ingredients | Percent (%) w/w |

Part A (Drug Coated Granules)

| | |
|---|---|
| Dapagliflozin base | 4.05 |
| Polyethylene glycol (PEG 400) | 8.09 |
| Lactose anhydrous | 20.23 |
| Microcrystalline cellulose | 40.45 |

Part B

| | |
|---|---|
| Drug Coated Granules of Part A | 72.82 |
| Microcrystalline cellulose | 18.20 |
| Crospovidone | 4.05 |
| Colloidal silicon dioxide | 1.21 |
| Magnesium stearate | 0.81 |

Film coating

| | |
|---|---|
| Opadry ® coating | 2.91 |

### Procedure:

1. Lactose anhydrous and intragranular microcrystalline cellulose were blended.
2. Dapagliflozin base was added to polyethylene glycol (PEG 400) while stirring to form a single phase solution.
3. The material of step 1 was granulated with the solution of step 2, followed by drying of the granules.
4. The dried granules of step 3 were blended with extragranular microcrystalline cellulose and crospovidone, followed by the addition of colloidal silicon dioxide and magnesium stearate.
5. The blend of step 4 was compressed into tablets.
6. The tablets of step 5 were coated with Opadry®.

### Example 4

| | |
|---|---|
| Ingredients | Percent (%) w/w |

Part A (Drug Coated Granules)

| | |
|---|---|
| Dapagliflozin base | 4.05 |
| Hydroxypropylmethyl cellulose | 4.05 |
| Isopropyl alcohol | q.s. |
| Methylene chloride | q.s. |
| Lactose anhydrous | 20.23 |
| Microcrystalline cellulose | 42.46 |

Part B

| | |
|---|---|
| Drug coated granules of part A | 70.79 |
| Microcrystalline cellulose | 19.82 |
| Crospovidone | 4.05 |
| Colloidal silicon dioxide | 1.46 |
| Magnesium stearate | 0.97 |

Film coating

| | |
|---|---|
| Opadry ® coating | 2.91 |

### Procedure:

1. Lactose anhydrous and intragranular microcrystalline cellulose were blended.
2. Hydroxypropylmethyl cellulose was dissolved in a solvent mixture (isopropyl alcohol:methylene chloride mixture (1:1.5 ratio)), followed by the dispersion of dapagliflozin base while stirring.
3. The material of step 1 was granulated with the dispersion of step 2, followed by drying of the granules.
4. The dried granules of step 3 were blended with extragranular microcrystalline cellulose and crospovidone, followed by the addition of colloidal silicon dioxide and magnesium stearate.
5. The blend of step 4 was compressed into tablets.
6. The tablets of step 5 were coated with Opadry®.

### Example 5

| | |
|---|---|
| Ingredients | Percent (%) w/w |

Intragranular

| | |
|---|---|
| Dapagliflozin base | 4.05 |
| Polyethylene glycol | 16.18 |
| Microcrystalline cellulose | 50.56 |
| Lactose anhydrous/monohydrate | 20.23 |
| Crospovidone | 4.05 |
| Colloidal silicon dioxide | 1.21 |
| Sodium stearyl fumarate | 0.81 |

Film coating

| | |
|---|---|
| Opadry ® coating | 2.91 |

### Procedure:

1. Sift and mix dapagliflozin base, polyethylene glycol, and half of the colloidal silicon dioxide.
2. Transfer the material of step 1 to a hot-melt extruder to obtain extruded material, followed by milling.
3. Blend microcrystalline cellulose, crospovidone, lactose monohydrate, and the remaining half of the colloidal silicon dioxide.
4. Blend the material of step 2 and step 3, followed by the addition of sodium stearyl fumarate.
5. Compress the blend of step 4 into tablets.
6. Film coat the tablets of step 5 with Opadry®.

### Example 6

| | |
|---|---|
| Ingredients | Percent (%) w/w |

Intragranular

| | |
|---|---|
| Dapagliflozin base | 4.05 |
| Copovidone | 16.18 |
| Microcrystalline cellulose | 50.56 |
| Lactose anhydrous/monohydrate | 20.23 |
| Crospovidone | 4.05 |
| Colloidal silicon dioxide | 1.21 |
| Magnesium stearate | 0.81 |

Film coating

| | |
|---|---|
| Opadry ® coating | 2.91 |

### Procedure:

1. Sift and mix dapagliflozin base, copovidone, and half of the colloidal silicon dioxide.
2. Transfer the material of step 1 to a hot-melt extruder to obtain extruded material, followed by milling.
3. Blend microcrystalline cellulose, crospovidone, lactose monohydrate, and the remaining half of the colloidal silicon dioxide.
4. Blend the material of step 2 and step 3, followed by the addition of magnesium stearate.
5. Compress the blend of step 4 into tablets.
6. Film coat the tablets of step 5 with Opadry®.

We claim:

1. A pharmaceutical composition comprising a solid dispersion of dapagliflozin and one or more pharmaceutically acceptable excipients.

2. The pharmaceutical composition according to claim 1, wherein the solid dispersion comprises dapagliflozin and a carrier.

3. The pharmaceutical composition according to claim 2, wherein the carrier comprises a hydrophilic polymer and, optionally, one or more pharmaceutically acceptable excipients.

4. The pharmaceutical composition according to claim 2, wherein the weight ratio of the dapagliflozin to the carrier is about 1:0.1 to about 1:100.

5. The pharmaceutical composition according to claim 1, wherein the pharmaceutically acceptable excipients are selected from the group comprising diluents, binders, disintegrants, lubricants, glidants, stabilizers, surfactants, solubility enhancers, coloring agents, flavoring agents, and mixtures thereof.

6. The pharmaceutical composition according to claim 1, wherein the solid dispersion is prepared by a solvent method.

7. The pharmaceutical composition according to claim 1, wherein the solid dispersion is prepared by a hot-melt extrusion method.

8. The pharmaceutical composition according to claim 1, wherein the composition is in the form of caplets, pills, mini-tablets, granules, pellets, tablets, or capsules.

9. The pharmaceutical composition according to claim 1, wherein the composition is film-coated.

10. A process for the preparation of the pharmaceutical composition according to claim 2, wherein the process comprises:
(a) dissolving a carrier in a solvent to obtain a solution;
(b) dispersing dapagliflozin in the solution of step (a) to obtain a dispersion;
(c) blending a diluent and, optionally, a binder;
(d) granulating the blend of step (c) with the dispersion of step (b) to obtain drug coated granules, followed by drying of the granules;
(e) blending the dried granules of step (d) with a disintegrant, a lubricant, and/or a glidant; and
(f) compressing the lubricated granules of step (e) into tablets or filling into capsules

11. The process according to claim 10, wherein the process of forming the granules consists essentially of:
(a) dissolving the carrier in the solvent to obtain the solution;
(b) dispersing dapagliflozin in the solution of step (a) to obtain the dispersion;
(c) blending the diluent and the optional binder; and
(d) granulating the blend of step (c) with the dispersion of step (b) to obtain the drug coated granules.

12. The process according to claim 10, wherein the process of forming the granules consists of:
(a) dissolving the carrier in the solvent to obtain the solution;
(b) dispersing dapagliflozin in the solution of step (a) to obtain the dispersion;
(c) blending the diluent and the optional binder; and
(d) granulating the blend of step (c) with the dispersion of step (b) to obtain drug coated granules.

13. A process for the preparation of the pharmaceutical composition of claim 2, wherein the process comprises:
(a) dissolving dapagliflozin in a carrier;
(b) blending a diluent and, optionally, a binder;
(c) granulating the blend of step (b) with the solution of step (a) to obtain drug coated granules, followed by drying of the granules;
(d) blending the dried granules of step (c) with a disintegrant, a lubricant, and/or a glidant; and
(e) compressing the lubricated granules of step (d) into tablets or filling into capsules.

14. The process according to claim 13, wherein the process of forming the granules consists essentially of:
(a) dissolving the dapagliflozin in the carrier;
(b) blending the diluent and the optional binder; and
(c) granulating the blend of step (b) with the solution of step (a) to obtain drug coated granules.

15. The process according to claim 13, wherein the process of forming the granules consists of:
(a) dissolving the dapagliflozin in the carrier;
(b) blending the diluent and the optional binder; and
(c) granulating the blend of step (b) with the solution of step (a) to obtain drug coated granules.

16. A process for the preparation of the pharmaceutical composition according to claim 2, wherein the process comprises:
(a) blending dapagliflozin, a carrier, and a glidant in a rapid mixer granulator;
(b) loading the blend of step (a) in a hot-melt extruder to obtain extrudates;
(c) milling the extrudates of step (b) and blending the milled extrudates with a diluent, a binder, a disintegrant, a glidant, and/or a lubricant; and
(d) compressing the blend of step (c) into tablets or filling into capsules.

17. The process according to claim 16, wherein the process of forming extrudates consists essentially of:
(a) blending dapagliflozin, the carrier, and the glidant in a rapid mixer granulator; and
(b) loading the blend of step (a) into a hot-melt extruder to obtain extrudates.

18. The process according to claim 16, wherein the process of forming extrudates consists of:
(a) blending dapagliflozin, the carrier, and the glidant in a rapid mixer granulator; and
(b) loading the blend of step (a) into a hot-melt extruder to obtain extrudates.

19. A method of treating diabetes by administering the pharmaceutical composition according to claim 1.
